(19) United States
(12) Patent Application Publication
(10) Pub. No.: US 2022/0240554 A1
(43) Pub. Date: Aug. 4, 2022

(54) Method for Stabilizing Rice Bran with Complex Enzyme

(71) Applicant: Sericultural & Agri-Food Research Institute Guangdong Academy of Agricultural Sciences, Guangzhou City (CN)

(72) Inventors: Guang Liu, Guangzhou City (CN); Mingwei Zhang, Guangzhou City (CN); Yuanyuan Deng, Guangzhou City (CN); Zhencheng Wei, Guangzhou City (CN); Yan Zhang, Guangzhou City (CN); Xiaojun Tang, Guangzhou City (CN); Ping Li, Guangzhou City (CN); Pengfei Zhou, Guangzhou City (CN); Zhihao Zhao, Guangzhou City (CN); Zhiming Wang, Guangzhou City (CN); Jiajia Wang, Guangzhou City (CN); Na Liao, Guangzhou City (CN)

(21) Appl. No.: 17/587,330

(22) Filed: Jan. 28, 2022

(30) Foreign Application Priority Data
Feb. 1, 2021 (CN) 2021101375809

(51) Int. Cl. A23L 7/10; A23L 3/16; A23L 3/3571; A23L 7/104

(52) U.S. Cl. CPC A23L 7/115; A23L 7/107; A23L 3/3571; A23L 3/16

(57) ABSTRACT

A method for stabilizing rice bran with complex enzyme involving enzymatic hydrolysis combined with thermal processing to stabilize rice bran, includes compounding at least one of glycosyl hydrolases such as cellulase, hemicellulase and alpha amylase into a complex enzyme solution, and performing enzymatic hydrolysis on the rice bran with the complex enzyme solution, and after enzymatic hydrolysis, performing thermal processing and inactivating enzyme treatment on rice bran using the moist-heat method, microwave method and/or extrusion expansion method, to prepare the stabilized rice bran. From the perspective of reducing bound lipase from rice bran, the glycosyl hydrolase is used to catalyze conversion of bound lipase in rice bran into free lipase, thereby effectively improving inactivation efficiency of rice bran lipase. The rice bran prepared using the method has characteristics of low residual activity of lipase and long shelf life, and may be directly applied to industries such as food and cosmetics.

### TECHNICAL FIELD

The application belongs to the field of grain processing, and in particular relates to a method for stabilizing rice bran with complex enzyme.

### BACKGROUND

Rice bran is a by-product produced during rice processing, accounting for about 8% of the rice weight. Rice bran consists of the mixture of seed coat, perisperm, aleurone layer and embryo of rice, and rice bran is rich in nutrients and active ingredients such as protein, oil, dietary fiber, vitamins, minerals, oryzanol, γ-aminobutyric acid and phenols. Therefore, rice bran is known as the “treasure trove of natural nutrition” and is a by-product for development.

In China, the annual output of rice bran is about 13 million tons. However, the rice bran has not been effectively developed and utilized for a long time. Only less than 10% of the rice bran is used to extract oil or high-value nutrients, while more than 90% of the rice bran is used for livestock feed, resulting in a great waste of resources. Accordingly, under the background of promoting food security, it is of great significance to strengthen the food utilization of rice bran.

The biggest difficulty that restricts the development and utilization of rice bran is that rice bran is difficult to store and prone to rancidity and deterioration. Besides a large amount of oil, rice bran also contains active lipase and lipoxygenase. Under the action of enzyme, oil is rapidly hydrolyzed, oxidized and produces a lot of fatty acids, and it smells bitter, numb and pungent, which leads to the rapid acidification of rice bran and the deterioration of organoleptic quality. Thus rice bran should be properly stabilized before effectively used.

At present, methods are used to stabilize rice bran, such as low-temperature storage, radiation treatment, microwave treatment, extrusion expansion, moist-heat treatment, protease hydrolysis and so on. Although these methods may inactivate lipase activity to a certain extent and prolong its rancidity time, these methods still couldn't completely inactivate lipase. Lipases in rice bran may be classified into free state and bound state. Conventional protease treatment or heat treatment have good inactivation effects on free lipase, but poor effects on bound lipase, which makes it difficult to completely overcome the rancidity and deterioration drawbacks of rice bran in the prior art and affects the exploitation of high added value of rice bran.

### SUMMARY

In order to overcome the shortcomings of the prior art, the application aims to provide a method for stabilizing rice bran with complex enzymes. The method mainly involves enzymatic hydrolysis combined with thermal processing to treat rice bran, specifically including compounding one or more of glycosyl hydrolases such as cellulase, hemicellulase and alpha amylase into a complex enzyme solution, and performing enzymatic hydrolysis on the rice bran with the complex enzyme solution, and after the enzymatic hydrolysis, thermal processing and inactivating enzyme treatment on the rice bran is carried out by using one or more of the moist-heat method, microwave method or extrusion expansion method, so as to prepare the stabilized rice bran.

The purpose of the application can be achieved by the following technical schemes:

a method for stabilizing rice bran with complex enzyme includes the following steps:

(1) dissolving glycosyl hydrolases with distilled water into 5-25 percent by weight (% w/w) glycosyl hydrolase solution;

(2) preparing fresh rice bran, taking the glycosyl hydrolase solution being 2-8% w/w of the rice bran, uniformly spraying the taken glycosyl hydrolase solution into the rice bran by using an atomizing device, and continuously stirring;

(3) incubating the rice bran to be enzymatically hydrolyzed in an environment with a temperature of 50-80° C. and a humidity of 50-70% for 3-5 hours, and turning the rice bran every 15-30 minutes (preferably every 30 minutes) during the incubating process; and

(4) carrying out thermal processing and inactivation enzyme treatment on the rice bran after enzymolysis to obtain stabilized rice bran.

In an embodiment, the glycosyl hydrolases include at least one of cellulase, hemicellulase and alpha amylase.

Preferably, the glycosyl hydrolases are the cellulase, the hemicellulase and the alpha amylase.

Preferably, the weight ratio of cellulase:hemicellulase:alpha amylase is 50-70:20-40:5-20.

Further, the weight ratio of cellulase:hemicellulase:alpha amylase is 55-65:25-35:10-15, and even further 55-65:25-30:10-15.

In an embodiment, the enzymatic activity of the cellulase is 5000 U/g, the enzymatic activity of hemicellulase is 3000 U/g, and the enzymatic activity of alpha amylase is 10000 U/g.

Preferably, the concentration of the glycosyl hydrolase solution in step (1) is 10-20% w/w.

Preferably, in step (2), the taken glycosyl hydrolase solution is 3-6% w/w of the rice bran.

Preferably, in step (2), the time for the continuously stirring is 20-40 minutes, and further 25-35 minutes.

Preferably, in step (3), the incubation is carried out in an environment with a temperature of 60-70° C. and a humidity of 55-65% for 3.5-4.5 hours.

Preferably, in step (4), at least one of a moist-heat method, a microwave method and an extrusion expansion method is used to perform the thermal processing and inactivation enzyme treatment on the rice bran after enzymolysis.

In an embodiment, the treatment conditions of the moist-heat method are 121° C. for 10-30 minutes, and further 121° C. for 10-15 minutes.

In an embodiment, the treatment conditions of the microwave method are 450-500 W (watts) for 2-5 minutes, and further 500 W for 2-5 minutes.

In an embodiment, the treatment conditions of the extrusion expansion method are barrel temperature of 120-140° C. and screw rotation speed of 150-250 r/min (revolutions per minute); and further, the barrel temperature is 130° C. and the screw speed is 200 r/min.

After undergoing above steps, rice bran with higher inactivation rate of lipase activity and better stability could be obtained.

Compared with the prior art, the application may have the following advantages and effects:

(1) the application provides a method for stabilizing rice bran with complex enzyme, which may effectively improve the efficiency of thermal treatment to inactivate rice bran lipase, prolong the shelf life of rice bran, and promote the use of rice bran in food and cosmetics industries.

(2) According to the application, the rice bran stabilization routine of enzymatic hydrolysis of lipase by protease is broken, and in order to reduce the bound lipase of rice bran, glycosyl hydrolase (cellulase, amylase, etc.) is used to catalyze the transformation of bound lipase in rice bran to free lipase, thus effectively improving the inactivation efficiency of rice bran lipase. Additionally the idea of stabilizing rice bran with enzyme method is provided. The rice bran prepared has the characteristics of low lipase residual activity, long shelf life and so on, and may be directly applied to foodstuff, cosmetics and other industries.

### BRIEF DESCRIPTION OF DRAWINGS

FIG. 1 shows the influence of different glycosyl hydrolases on percentages of free lipase and bound lipase activity in rice bran.

FIGS. 2A and 2B show percentages of free lipase and bound lipase activity in different treatment groups and the influence of different treatment groups on lipase residual activity. In particular, FIG. 2A shows the percentages of free lipase and bound lipase in different treatment groups, and FIG. 2B shows the influence of different treatment methods on the residual activity of lipase. The control group is unstabilized rice bran without complex enzyme treatment, the sample group 1 is stabilized rice bran without complex enzyme treatment, and the sample group 2 is stabilized rice bran treated with complex enzyme treatment. Different lowercase letters indicate significant differences (p<0.05).

FIGS. 3A and 3B show percentages of free lipase and bound lipase activity in different treatment groups and the influence of different treatment groups on the acid value of rice bran. In particular, FIG. 3A shows the percentages of free lipase and bound lipase in different treatment groups, and FIG. 3B shows the influence of different treatment methods on the acid value of rice bran. The control group is unstabilized rice bran without complex enzyme treatment, the sample group 1 is stabilized rice bran without complex enzyme treatment, and the sample group 2 is stabilized rice bran treated with complex enzyme treatment. Different lowercase letters indicate significant differences (p<0.05).

FIGS. 4A and 4B show the influence of different treatments on the residual activity and acid value of rice bran lipase. The control group is unstabilized rice bran without complex enzyme treatment, the sample group 1 is stabilized rice bran without complex enzyme treatment, and the sample group 2 is stabilized rice bran treated with complex enzyme treatment. Different lowercase letters indicate significant differences (p<0.05).

### DETAILED DESCRIPTION OF EMBODIMENTS

The application will be described in further details below with embodiments and drawings, but embodiments of the invention are not limited thereto.

For the process parameters not specifically indicated, it may be carried out with reference to conventional technology. The rice bran used in the embodiment of the application is provided by Guangdong Haina Agriculture Co., Ltd., and the cellulase, hemicellulase and alpha amylase used are purchased from Sigma Company, and the enzyme activities are 5000 U/g, 3000 U/g and 10000 U/g, respectively.

### Embodiment 1

Cellulase, hemicellulase or α-amylase are respectively prepared into a single enzyme solution with a concentration of 10% w/w. Fresh rice bran is prepared and then is added with the single enzyme solution being 3% w/w of the weight of rice bran, the single enzyme solution is sprayed into the rice bran uniformly by using an atomizing device, and continuous stirring is carried out for 35 minutes. Subsequently, the rice bran to be enzymatically hydrolyzed is incubated in an environment with a temperature of 70° C. and a humidity of 65% for 3.5 hours, and the raw materials are turned every 30 minutes during the incubation. After the enzymolysis, the free lipase in rice bran is repeatedly extracted with 50 mM phosphate buffer, and the total lipase and free lipase activity in rice bran are determined, and the bound lipase and free lipase activity are calculated respectively.

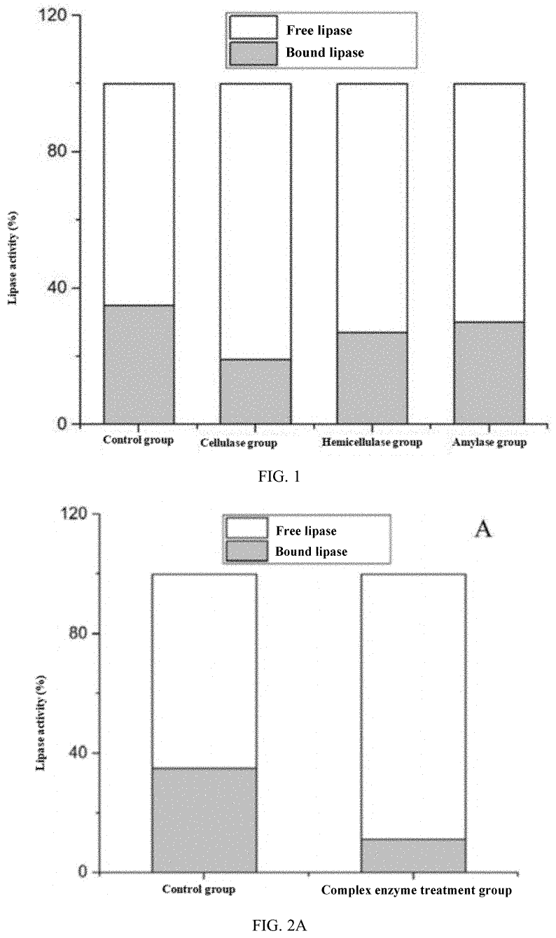

It can be seen from the control group in FIG. 1 that there are two types of lipase in rice bran: free state and bound state, and the activity ratios are 65% and 35% respectively. After treatment with cellulase, hemicellulase or a amylase, the percentages of extractable free lipase activity in rice bran increase to 81%, 73% and 70% respectively, while the percentages of bound lipase activity decrease to 19%, 27% and 30% respectively. The results show that using a single glycosyl hydrolase could increase the percentage of free lipase activity in rice bran, but the effects of three different glycosyl hydrolases are compared, it is found that cellulase is more effective in catalyzing the transformation from bound lipase to free lipase in rice bran.

### Embodiment 2

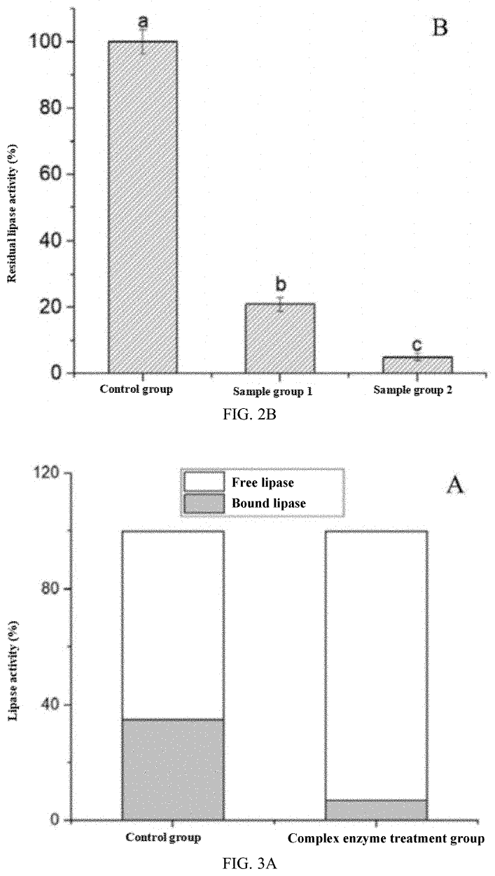

Cellulase, hemicellulase and a amylase are compounded and mixed according to 65:25:10 parts by weight, and dissolved in distilled water to form a complex enzyme solution with a concentration of 10% w/w. Fresh rice bran is prepared and then is added with the complex enzyme solution being 3% w/w of the rice bran, spraying the complex enzyme solution into the rice bran evenly with an atomizing device, and keep stirring for 35 minutes. Subsequently, the rice bran to be enzymatically hydrolyzed is incubated in an environment with a temperature of 70° C. and a humidity of 65% for 3.5 hours, and the raw materials are turned every 30 minutes during the incubation. After the enzymolysis, the free lipase in rice bran is repeatedly extracted with 50 mM phosphate buffer, the total lipase and free lipase activity in rice bran are determined, and the bound lipase and free lipase activity are calculated respectively. The rice bran is further stabilized by adopting the moist-heat method, and the moist-heat treatment conditions are 121° C. for 15 minutes to prepare the stabilized rice bran. The residual activity of lipase in rice bran is determined by alkali titration method. The rice bran without complex enzyme treatment is used as positive control (recorded as sample group 1), the rice bran without complex enzyme treatment and moist-heat treatment is the negative control (the control group), and the determination is repeated for three times. The results are shown in FIGS. 2A and 2B.

It can be seen from the control group in FIG. 2A that there are two types of lipase in rice bran: free state and bound state, and the activity ratios are 65% and 35% respectively. After complex enzyme treatment, the percentage of extractable free lipase activity in rice bran increases to about 90%, while the percentage of bound lipase activity decreases to about 10%. The results show that enzymatic hydrolysis with complex enzymes could effectively promote the transformation from bound lipase to free lipase in rice bran, and the catalytic transformation effect is better than that of single enzyme treatment, and indicates that complex enzyme treatment has synergistic effects.

In addition, in FIG. 2B, the effect of moist-heat treatment on the activity of rice bran lipase residual in different treatment groups is compared; as can be seen from the drawing, compared with the control group, the lipase activity of sample group 1 and sample group 2 after wet heat treatment is significantly reduced, and the residual lipase activity is 21% and 5%, respectively; comparing the sample group 1 and the sample group 2, it may be found that the lipase activity inactivation rate of the sample group 2 treated with the complex enzyme is higher, indicating that the rice bran lipase treated with the complex enzyme is more sensitive to heat and easier to inactivate.

These results show that the enzymatic hydrolysis of rice bran by complex enzyme may effectively promote the conversion of bound lipase to free lipase in rice bran, and increase the activity ratio of free lipase in rice bran, which is beneficial to the inactivation efficiency of rice bran lipase by thermal processing.

### Embodiment 3

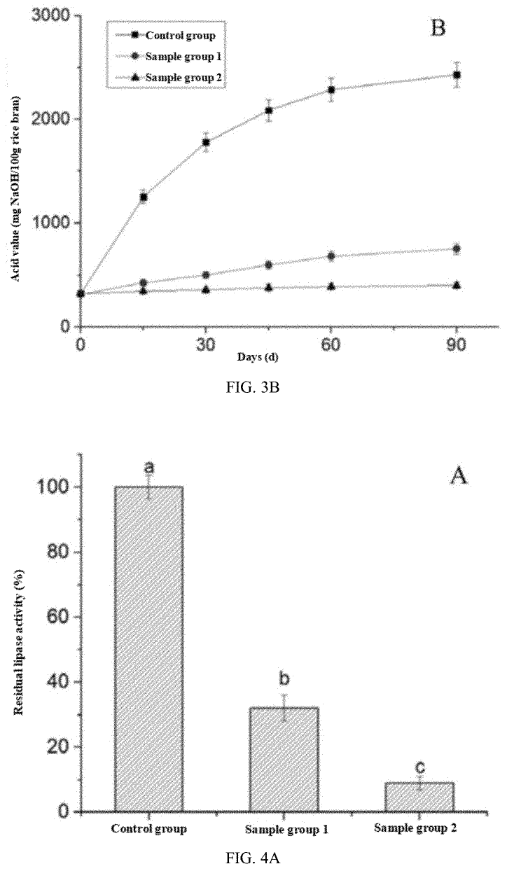

Cellulase, hemicellulase and a amylase are compounded and mixed according to 60:30:10 parts by weight, and dissolved in distilled water to form a complex enzyme solution with a concentration of 15% w/w. Fresh rice bran is prepared and then is added with the complex enzyme solution being 4.5% w/w of the weight of the rice bran, spraying the complex enzyme solution into the rice bran evenly with an atomizing device, and keeping stirring for 30 minutes. Subsequently, the rice bran to be enzymatically hydrolyzed is incubated in an environment with a temperature of 65° C. and a humidity of 60% for 4 hours, and the raw materials are turned every 30 minutes during the incubation. After the enzymolysis, the free lipase in rice bran is repeatedly extracted with 100 mM phosphate buffer, the total lipase and free lipase activity in rice bran are determined, and the bound lipase and free lipase activity are calculated respectively. The rice bran is further stabilized by microwave heating at 500 W for 2 minutes, and the stabilized rice bran is prepared. The rice bran is stored at 37° C. for 3 months, and its acid value is measured. The rice bran without complex enzyme treatment is used as positive control (recorded as sample group 1), and the rice bran without complex enzyme treatment and microwave treatment is used as negative control (recorded as control group), and the results were repeated for 3 times, as shown in FIG. 3. The practice is repeated for 3 times, and the results are shown in FIGS. 3A and 3B.

It can be seen from the control group in FIG. 3A that there are two types of lipase in rice bran: free state and bound state, and the activity ratios are 65% and 35%, respectively. After complex enzyme treatment, the proportion of extractable free lipase activity in rice bran increases to 93%, while the proportion of bound lipase activity decreases to about 7%. Compared with the control group, the percentage of free lipase activity increases by 28%, which is related to the fact that complex enzymes may effectively promote the transformation from bound lipase to free lipase in rice bran, and the catalytic transformation effect is better than that of single enzyme treatment, that is, complex enzyme treatment has synergistic effects.

In addition, the change results of acid value of rice bran in different treatment groups during storage at 37° C. for 3 months are shown in FIG. 3B. It can be seen from the figure that the acid value of rice bran in the control group increases significantly with storage time, and after storage for 3 months, its acid value reaches 2490 mg NaOH/100 g rice bran, which is nearly 7 times higher than that on the 0th day. Compared with the sample group 1 and sample group 2, the acid value of rice bran after 90 days of storage increases by 140% and 25% respectively, that is, the rice bran treated with complex enzyme has better storage stability.

These results show that enzymatic hydrolysis of rice bran with complex enzymes could effectively promote the transformation of bound lipase to free lipase in rice bran, thus promoting the inactivation efficiency of rice bran lipase by thermal processing and improving the storage stability and shelf life of rice bran.

### Embodiment 4

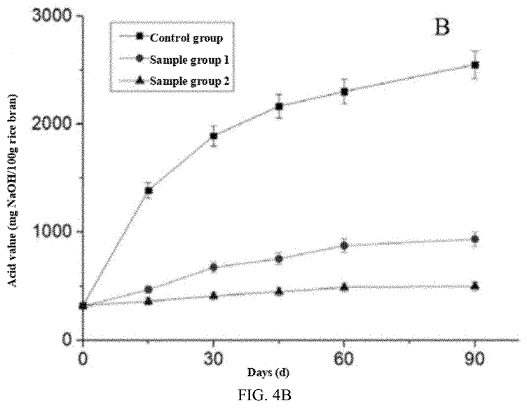

Cellulase, hemicellulase and a amylase are compounded and mixed according to 55:30:15 parts by weight, and dissolved in distilled water to form a complex enzyme solution with a concentration of 20% w/w. Fresh rice bran is prepared and then is added with the complex enzyme solution being 6% w/w of the weight of the rice bran, spraying the complex enzyme solution into the rice bran evenly with an atomizing device, and keeping stirring for 25 minutes. Subsequently the rice bran to be enzymatically hydrolyzed is incubated in an environment with a temperature of 60° C. and a humidity of 55% for 4.5 hours, and the raw materials are turned every 30 minutes during the incubation. After the enzymolysis, the rice bran is stabilized by extrusion expansion under the conditions of barrel temperature of 130° C. and screw speed of 200 r/min, and the stabilized rice bran is prepared. The activity of rice bran lipase residual is determined by alkaline titration method, and the rice bran is stored at 37° C. for 3 months to determine the change of its acid value. The rice bran without complex enzyme treatment is used as positive control (recorded as sample group 1), and the rice bran without complex enzyme treatment and extrusion expansion treatment is used as negative control (recorded as control group), and the practice is repeated for 3 times, as shown in FIGS. 4A and 4B.

In FIG. 4A, the influence of extrusion expansion treatment on the residual lipase activity of rice bran in different treatment groups is compared. It can be seen from the figure that compared with the control group, the lipase activity of sample group 1 and sample group 2 after extrusion treatment is significantly reduced, and the residual lipase activity is 32% and 9% respectively. Comparing the sample group 1 and the sample group 2, it can be found that the lipase activity inactivation rate of the sample group 2 treated with the complex enzyme is higher, indicating that the rice bran lipase treated with the complex enzyme is more sensitive to heat and easier to inactivate.

In addition, the change results of acid value of rice bran in different treatment groups during storage at 37° C. for 3 months are shown in FIG. 4B. It can be seen from the figure that the acid value of rice bran in the control group increases significantly with storage time, and after storage for 3 months, its acid value reached 2550 mg NaOH/100 g rice bran, which is 7 times higher than that on the 0 th day. Compared with sample group 1 and sample group 2, the acid value of rice bran after 90 days of storage increases by 197% and 55% respectively, that is, the rice bran treated with complex enzyme has better storage stability.

These results show that enzymatic hydrolysis of rice bran with complex enzymes could effectively improve the inactivation efficiency of rice bran lipase and prolong the storage stability and shelf life of rice bran.

The above embodiments are preferred embodiments of the application, but the application is not limited by the above illustrated embodiments. Any other changes, modifications, substitutions, combinations and simplifications that do not deviate from the spirit and principle of the application should be equivalent replacement solutions, which should fall in the scope of protection of the application.

What is claimed is:

1. A method for stabilizing rice bran with complex enzyme, comprising the following steps:
(1) dissolving glycosyl hydrolases with distilled water into a 5-25 percent by weight (% w/w) of glycosyl hydrolase solution, wherein the glycosyl hydrolases comprise at least one of cellulase, hemicellulase and alpha amylase;
(2) preparing fresh rice bran, taking the glycosyl hydrolase solution being 2-8% w/w of the rice bran, uniformly spraying the taken glycosyl hydrolase solution into the rice bran, and continuously stirring;
(3) incubating the rice bran to be enzymatically hydrolyzed in an environment with a temperature of 50-80° C. and a humidity of 50-70% for 3-5 hours, and turning the rice bran every 15-30 minutes during the incubating process; and
(4) carrying out thermal processing and inactivation enzyme treatment on the rice bran after enzymolysis to obtain stabilized rice bran.

2. The method for stabilizing rice bran with complex enzyme according to claim 1, wherein the glycosyl hydrolases are the cellulase, the hemicellulase and the alpha amylase.

3. The method for stabilizing rice bran with complex enzyme according to claim 2, wherein a weight ratio of cellulase:hemicellulase:alpha amylase is 50-70:20-40:5-20.

4. The method for stabilizing rice bran with complex enzyme according to claim 3, wherein the weight ratio of cellulase:hemicellulase:alpha amylase is 55-65:25-35:10-15.

5. The method for stabilizing rice bran with complex enzyme according to claim 1, wherein an enzymatic activity of the cellulase is 5000 U/g, an enzymatic activity of the hemicellulase is 3000 U/g, and an enzymatic activity of the alpha amylase is 10000 U/g.

6. The method for stabilizing rice bran with complex enzyme according to claim 1, wherein a concentration of the glycosyl hydrolase solution in step (1) is 10-20% w/w; and
in step (2), the taken glycosyl hydrolase solution is 3-6% w/w of the rice bran.

7. The method for stabilizing rice bran with complex enzyme according to claim 1, wherein in step (2), a time for the continuously stirring is 20-40 minutes; and
in step (3), the incubating is carried out in the environment with the temperature of 60-70° C. and the humidity of 55-65% for 3.5-4.5 hours.

8. The method for stabilizing rice bran with complex enzyme according to claim 1, wherein in step (4), at least one of a moist-heat method, a microwave method and an extrusion expansion method is used to perform the thermal processing and inactivation enzyme treatment on the rice bran after enzymolysis.

9. The method for stabilizing rice bran with complex enzyme according to claim 8, wherein treatment conditions of the moist-heat method are 121° C. for 10-30 minutes;
treatment conditions of the microwave method are 450-500 W for 2-5 minutes; and
treatment conditions of the extrusion expansion method are barrel temperature of 120-140° C. and screw rotation speed of 150-250 r/min.

10. The method for stabilizing rice bran with complex enzyme according to claim 9, wherein the treatment conditions of the moist-heat method are 121° C. for 10-15 minutes;
the treatment conditions of the microwave method are 500 W for 2-5 minutes; and
the treatment conditions of the extrusion expansion method are barrel temperature of 130° C. and screw rotation speed of 200 r/min.
